(19) United States
(12) Patent Application Publication
(10) Pub. No.: US 2009/0118378 A1
(43) Pub. Date: May 7, 2009

(54) Technical information for a formula of a natural facial moisturizing cream for daytime use which contains a propylene glycol extract of plant specie solanum tuberosum

(76) Inventor: Valerie Joycelyne Barbier, Lima / Miraflores (PE)

Correspondence Address: DLA PIPER LLP (US), 4365 EXECUTIVE DRIVE, SUITE 1100, SAN DIEGO, CA, 92121-2133, US

(21) Appl. No.: 11/979,376

(22) Filed: Nov. 2, 2007

(51) Int. Cl. A61K 31/047; A61P 17/00

(52) U.S. Cl. 514/724

(57) ABSTRACT

This invention relates to facial moisturizing cream used for daytime use in the cosmetics industry; it provides active antioxidants present in the extract of the plant species Solanum Tuberosum from the Peruvian Andes; which promotes the health of the skin and helps prevent the appearance of wrinkles to counteract the damaging effects of free radicals formed by environmental stress. This formula has been designed to be quickly absorbed while protecting and moisturizing the skin, providing greater elasticity and smoothness without leaving any annoying oily layer.

### BACKGROUND OF THE INVENTION

1. Field of the Invention

This invention is a moisturizing facial cream for the cosmetic industry with active antioxidants contains in the vegetable extract of the species of Solanum tuberosum from the Peruvian highlands to care for the skin and help prevent wrinkles by counteracting the negative effects of the elements. It is absorbed quickly due to its special formula, protecting and moisturizing the skin, while giving it better elasticity and a smooth feel without leaving an oily film.

2. Background Information

The inventions, known in the market in the form of creams, such as Nivea, Pond's, Unique, contain excipients from petroleum by-products which can cause damage and irritation to the skin.

In pre-determined circumstances, the production of free radicals can enlarge in form without control, a situation known as oxidative stress.

The current concept details the existence of the imbalance between the velocities of production and the destruction of toxic molecules that give rise to an increase and cellular concentration of free radicals.

The evolution has been such that the cells have developed protection mechanisms against the harmful effect of free radicals; this is essentially a defense mechanism constituted by the antioxidant agents.

Those can be enzymatic mechanism, called endogenous antioxidants—which include the superoxide enzymes dismutase, catalase, glutathione peroxides, glutathione and the coenzyme Q—or the exogenous antioxidants, which admit into the organism throughout the via of the aliments.

When the exogenous antioxidants arrive to the cells, they can settle in their membranes and protect them from the lipoperoxidation. That is the case for vitamin E and carotene.

Those non enzymatic antioxidants react with free radicals and change their structure; in essence, the free radicals are captured and neutralized and oxidized in the process.

The enzymatic antioxidants are metal-proteins which act directly on the free radicals or reactive metabolite giving non radicals products. The elements which form part of those proteins are selenium, copper, zinc, manganese, iron.

### Phenolic Compounds

The phenolic compounds constitute one group of micronutrients present in the plant kingdom, including a large group of chemicals which are considered secondary metabolites of plants with different chemical structures and activities, comprising more than 8,000 different compounds.

These compounds have traditionally been regarded as antinutrients, due to the adverse effect of one of their major components, the tannins, on the digestibility of the protein. However, it now has attracted recent interest in these compounds because of their antioxidant properties.

The distribution of phenolic compounds in the tissues and cells of plants varies considerably according to the type of chemical compound in question located in the interior of the cell or the cell wall.

Its main functions in plant cells are the one which act as an essential metabolite for the growth and production of plants, and as protective agents compared to the action of pathogens, being secreted as a defense mechanism.

The interest for the antioxidant activity of phenolic compounds is due to a potential technological and nutritional application. Thus, phenolic compounds act as natural antioxidants, so their presence in food poses a reduction in the need for the use of antioxidant additives; at the same time, healthier foods are obtained, and even can make it a functional element.

From a nutritional standpoint, this antioxidant activity is associated with its protective role in cardiovascular disease and in cancer as well as in aging processes which are being intensively studied through “in vivo” and “in vitro” trials.

This present invention is a moisturizing cream that contains a Propylene Glycol extract from the vegetable species Solatium Tuberosum, which contains antioxidants that protect against the harmful effects of the free radicals formed by the negative effects of the elements. Also, the base is a special one because, without preservatives, the cream softens the skin.

### The Properties of the Solanum Tuberosum

### Chemical Structure and Classification

Chemical point of view: The phenolic compounds are chemical substances which have an aromatic ring, a benzene ring, with one or more hydroxide groups including derivatives such as esters, methyl esters, glycosides, etc.

This nature of polyphenols ranges from simple molecules such as phenolic acids to highly polymerised compounds like tannins.

Those are present in the plants in a combined form with one or more sugar residues attached to the hydroxyl groups, though in some cases, it is possible to produce direct unions between a sugar molecule and a aromatic carbon.

Therefore, the most common way to find them in nature is in the form of glycosides, which are soluble in water and in organic solvents.

The sugars associated to the polyphenols can be monosaccharide, disaccharides or even oligosaccharide.

The compounds which are linked most often are: glucose, galactose, arabinose, rhamnose, xylose, glucuronic and galacturonic acids.

They can also be attached to carboxylic acids, organic acids, amines, lipids and other phenolic compounds.

According to E. Harbome, phenolic compounds can be grouped into different classes depending on their basic chemical structure, described a continuation those with a major nutritional interest:

Phenols, Phenolic Acids and Phenylacetic Acids

Within this group of phenols, simple as phenol, cresol, thymol and resorcinol, are widely distributed between all the vegetable species.

Similarly, the phenolic acids such as gallic, vainilline, phydroxibenzoic, and aldehydes such as vanillin, are also abundant in superior plants and ferns.

By contrast, there is little information in the scientific literature about the phenylacetic acids in plants.

Flavonoids

The flavonoids constitute the group the most important inside this classification, dividing in different sub classes with more than 5,000 compounds, the polyphenols being the most distributed in the plants,

The polyphenols substances of low molecular weight share the common skeleton of dyphenilpirans, two benzene rings linked through a pyrone ring or heterocyclic pyran.

This basic structure presents or allows a multitude of substitutions and variations in the pyrone ring leading to flavonols, flavones, flavanones, flavanololes, isoflavonoides, catechins, chalcones, dihidrocalcones, anthocyanidines, leucoantocianidines orflavanols and proanthocyanidine or condensed tannins (non-hydrolysable tannins).

Within these groups of flavones (p.e. apigenin, luteolin and diosmetin), flavonoids (p.e. quercetin, myrecitin and kampferol) and their glycosides are the most abundant compounds in plants.

Tannins

The tannins are water-soluble phenolic compounds with a molecular weight between 500 and 3000 D.

These compounds contain a larger number of hydroxyl groups, among other functional groups (1 to 2% D), thus being able to join with proteins and others macromolecules.

The tannins can be classified in two groups: hydrolysable tannins and non-hydrolysable or condensed tannins.

The condensed tannins have a core central nucleus a polyhydric alcohol like glucose, and hydroxyl groups that are esterified either partially or completely with gallic acid or with the hexahydroxydiphenyl acid, making the gallotannins and alegytannins, respectively.

Alter hydrolysis with acids, bases or certain enzymes, the gallotannins give glucose and gallic acid.

Results of the phenols and total flavonoids obtained for the specie SOLANUM TUBEROSUM

| | | | Total Flavonoids | |
|---|---|---|---|---|
| | Total Phenols | | Equiv | umol |
| | Umol equiv | | routine | equiv |
| | pirogalol/ | umol equiv | umol/mL | routine/ |
| | mL extract | pirog/g mp | extract | g mp |
| SOLANUM | 0.78 | 2.7 | 0.25 | 1.25 |
| TUBEROSUM/PG | | | | |

Results obtained at the spectrometer 517 nm for the free radicals with the extract of Solanum Tuberosum

| | | |
|---|---|---|
| Control with light | 0.381 | 0.3855 |
| Control with light | 0.39 | |

| | | | | RESULTS |
|---|---|---|---|---|
| | | | | CONTROL |
| | | | Average | LIGHT 5 mn |
| Propylene Glycol extract of | 0.355 | 0.333 | 0.344 | 10.77% |
| Solanium tuberosum | | | | |
| YANA Day normal to oily skin | 0.361 | 0.334 | 0.3475 | 9.86% |

### Flow of Diagram for the Preparation of the Propylene Glycol Extract of Solanum Tuberosum

clean the vegetable species of the extract of Solatium tuberosum
cut in cubes the potatoes
macerate during 7 days in a concentration of Propylene Glycol 1 kg of Solanum Tuberosum/5 liters of Propylene Glycol and filter.

### EXAMPLES OF THE INVENTION

### The Cream

a—Composition:

| | | |
|---|---|---|
| A. | Water | 80-95% p/p |
| B. | Cetyl Alcohol | 2-5% p/p |
| C. | Stearic Acid | 0.5-2% p/p |
| D. | Ethyl Alcohol | 0.5-2% p/p |
| E. | Sodium borate | 0.5-2% p/p |
| F. | Tragacanth gum | 0.2-2% p/p |
| G. | Propylene Glycol extract | 0.5-2% p/p |
| | of Solatium tuberosum | |

b—Method of Preparation:

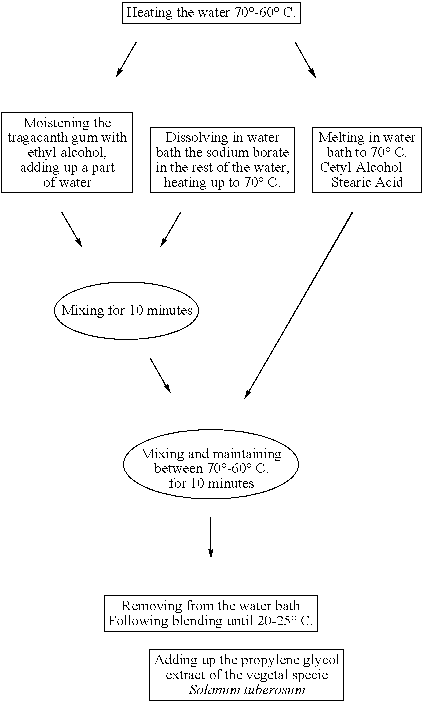

1) Melt in water bath the cetyl alcohol and stearic acid, at a temperature between 60° C. and 70° C.
2) Heat separately water and bring up to a temperature between 60° C. and 70° C.
3) Moisten the tragacanth gum with ethyl alcohol, and then add up a fraction of the water
4) Dissolve in the water bath the sodium borate with the remaining fraction of water, heating up between 60° C. and 70° C.
5) Add (3) (tragacanth gum/ethyl alcohol/water) in (4) (sodium borate/water) and mix for 10 minutes
6) Add (1) (cetyl alcohol/stearic acid) in (5) (tragacanth gum/ethyl alcohol/sodium borate/water), mix and maintain the temperature constant between 70°-60° C. during 10 minutes
7) Remove the preparation from the water bath and continue blending until homogenized preparation has cooled down (temperature between 20-25° C.)
8) Adding up the extract in % between 0.5% to 1%

### Technical Specifications:

Aspect: Pearly cream, of a white to a light yellow color. Visual

Smell: Light to Tragacanth

pH: 6.0-8.5−Temperature 25° C., sample diluted 1 in 10

Specific gravity: 0.800-1.100/Temperature 25° C., gravimetric

Dried residue: 3.0%-6.0%

Viscosity: Minimum 6000 cPs−Brookfield Viscometer, spindle 4, rapidity 12 rpm−Temperature 25° C.

### Microbial Content:

Total count of aerobes: Maximum 100 UFC/g

Combined count of fungi and yeast: Maximum 50 UFC/g

### Pathogenic:

Escherichia coli: Absent in 10 g

Salmonella sp: Absent in 10 g

Staphylococcus aureus: Absent in 10 g

Pseudomonas aeruginosa: Absent in 10 g

### Results of the Clinic Trial/Biologic

Evaluation of suitability and tolerance was made on a group of volunteers consisting of 45 women (Peruvian, French, Belgian, American, and Canadian), between the ages of 28 and 65 years of age, during a period of 21 days.

Results of the open questions on the characteristics of the cream:

54% (25 volunteers) reported that the cream penetrates very rapidly.
31% (14 volunteers) reported that the cream is not oily.
9% (4 volunteers) reported that the cream has a pleasant perfume.

Results of the open questions on the cosmetic effect of the cream on the face:

48% (22 volunteers) reported that 2-3 days later the skin was smoother.
20% (9 volunteers) reported that 2-3 days later the skin was more luxuriant, fresh.
17% (8 volunteers) reported that 2-3 days later the skin was more hydrated.
17% (8 volunteers) reported that 2-3 days later the skin was firmer.
9% (4 volunteers) reported that 2-3 days later the skin was more radiant.
7% (3 volunteers) reported that 2-3 days later the skin was finer.

### Comments from Customers

Cecilia Justo de Saavedra, Peruvian, 50 years, oily skin: “I love this cream because I have a very oily, shiny skin but with this cream I don't have any problem of shine!”

Yesenia, 32 years: “I think my skin is a little clearer. The cream has a smooth texture and smell. It really pleased me and I felt my skin more clean and clear.”

Hilde, 45 years: “It leaves the skin soft and elastic.”

Elsa, 66 years: “Firmer, hydrated and soft skin. Cream is easy to absorb and good texture that is refreshing when applied.”

Maria Teresa Cruz, Peruvian, 59 years, dry skin: “After using, my skin is better, firmer, the cream is absorbed very well.”

1-4. (canceled)

5. A composition comprising a propylene glycol extract of Solanum tuberosum.

6. A composition according to claim 5, wherein the propylene glycol extract of Solanum tuberosum is present in a concentration of about 0.5-2.0% by weight.

7. A composition according to claim 5, wherein the propylene glycol extract of Solanum tuberosum is present in a concentration of about 0.5-1.0% by weight.

8. A composition according to claim 5, wherein the composition is water-based.

9. A composition according to claim 5, further comprising a carrier.

10. A composition according to claim 8, further comprising an emollient, an emulsifier, a thickener, or a combination thereof.

11. A composition according to claim 8, further comprising one or more of the compounds selected from the group consisting of cetyl alcohol, stearic acid, ethyl alcohol, sodium borate, and tragacanth gum.

12. A composition according to claim 8, further comprising cetyl alcohol, stearic acid, ethyl alcohol, sodium borate, and tragacanth gum.

13. A method of manufacturing a cosmetic cream composition comprising:
adding a propylene glycol extract of Solanum tuberosum to a cosmetic cream preparation.

14. A method according to claim 13, wherein the cosmetic cream preparation comprises a blended mixture of water, cetyl alcohol, stearic acid, ethyl alcohol, sodium borate, and tragacanth gum.
